(19) United States
(12) Patent Application Publication
(10) Pub. No.: US 2010/0160450 A1
(43) Pub. Date: Jun. 24, 2010

(54) METHODS OF REDUCING 15-F2T-ISOP LEVELS IN MAMMALS

(76) Inventor: Eric Kuhrts, Bodega, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/525,502

(22) PCT Filed: Jan. 31, 2008
(86) PCT No.: PCT/US08/52692

§ 371 Date: Feb. 10, 2010

(51) Int. Cl. A61K 31/085; A61P 43/00

(52) U.S. Cl. 514/720

(57) ABSTRACT

Methods of reducing 15-F2t-IsoP levels in mammalian subjects are disclosed herein. In addition, methods of reducing or preventing oxidative stress and treating or preventing related diseases are disclosed.

### CROSS-REFERENCES TO RELATED APPLICATIONS

This application claims the benefit of U.S. Provisional Patent Application No. 60/887,578, filed Jan. 31, 2007, which is incorporated herein by reference in its entirety for all purposes.

### BACKGROUND OF THE INVENTION

Quantification of oxidative stress in vivo is an important issue that can be approached by measuring F₂-isoprostanes. F₂-isoprostanes are a complex family of compounds produced from arachidonic acid via a free-radical-catalyzed mechanism. The first demonstration that these compounds were produced in humans was shown in 1990 by Morrow et al., who reported the discovery of prostaglandin-F₂-like compounds generated by free-radical-induced peroxidation of arachidonic acid. Morrow et al., Proc Natl Acad Sci USA 87:9383-7 (1990). Because these compounds are isomeric to prostaglandins and have an F-type cyclopentane (prostane) ring, these compounds were termed F₂-isoprostanes. Since that time, F₂-isoprostanes have been used extensively as clinical markers of lipid peroxidation in vascular disorders. Cracowski, J., Chem Phys Lipids 128:75-83 (2004); Chiabrando et al., J Biol Chem 274:1313-9 (1999); Cracowski et al., Trends Pharmacol Sci 23:360-6 (2002). Several favorable attributes make measurement of F₂-isoprostanes a reliable biomarker of oxidative stress in vivo. Isoprostanes are stable in urine, where levels are present in detectable quantities, their formation increases in models of oxidant injury and are modulated by anti-oxidant status, but their levels are not affected by lipid content of the diet.

Among the F₂-isoprostanes, 15-F₂ₜ-IsoP, (9a,11a,15S-trihydroxy-(8b)-prosta-5Z,13E-dien-1-oic acid [CAS #27415-26-5] also known as 8-epi-prostaglandin F_{2α}, 8-epi-PGF_{2α}, 8-iso-PG F_{2α}, and also iPF_{2α}-III) is currently the most accurate clinical biomarker of lipid peroxidation and thus oxidative stress. Cracowski et al., Trends Pharmacol Sci 23:360-6 (2002). 15-F₂ₜ-IsoP is formed in vivo by the free radical catalyzed non-enzymatic peroxidation of arachidonic acid in cellular membranes and lipoproteins. The damaged lipid peroxide is excised from the cell wall into the serum and then excreted in urine. Once formed, 15-F₂ₜ-IsoP is chemically stable and can be accurately measured in serum or urine. See U.S. Pat. Nos. 5,858,696 and 5,700,654, each of which are herein incorporated by referenced in their entirety for all purposes. Therefore, 15-F₂ₜ-IsoP is a well-known and accurate means for assessing oxidative stress in mammals.

Oxidative stress is characterized by adverse effects occurring when the generation of reactive oxygen species (ROS) in a system exceeds a biological system's ability to neutralize and eliminate them. All forms of life maintain a reducing environment within their cells. The cellular redox environment is preserved by enzymes that maintain the reduced state through a constant input of metabolic energy. Disturbances in this normal redox state can cause toxic effects through the production of peroxides and free radicals that damage components of the cell such as lipids and DNA. In humans, oxidative stress is involved in many diseases, such as atherosclerosis, Alzheimer's disease, and aging.

Oxygen is reduced to water at the level of the mitochondrial respiratory chain in reactions catalyzed by cytochrome oxidase complexes. One molecule of dioxygen yields two molecules of water, by direct capture of four electrons and four protons. But oxygen can also undergo stepwise reduction, electron by electron. This leads to formation of highly toxic oxygen species, the reactive oxygen species (ROS), such as the superoxide radical anion (O₂⁻). By forming ROS, oxygen can aggressively compromise cell integrity.

Reactive oxygen species, and particularly free oxygen radicals, have short life spans. They interact with a wide variety of biological substrates such as nucleic acids, nucleotides, proteins, membrane lipids, and lipoproteins. ROS can produce breaks in deoxyribonucleic acid (DNA) and thus alter the genetic message. In the cytoplasm, ROS can transform molecules such as NADH or NADPH and thus alter the redox status of the cell and the activity of enzymes using these substrates. The action of ROS markedly modifies the primary, secondary, and tertiary structure of proteins, thereby denaturing them and forming insoluble aggregates (cell debris). Depolymerization of proteins such as collagen and elastin is a good example of the deleterious action of ROS. The protease inhibitor α-1-antitrypsin (which inhibits elastase and trypsin) is rapidly inactivated by free oxygen radicals. When red blood cells are in contact with ROS, their hemoglobin is altered and iron is released from the heme thereby increasing hemolysis.

Membrane phospholipids are essential constituents of cell architecture. They contain polyunsaturated fatty acids (PUFA), favored targets of free oxygen radicals. The result is a major alteration of membrane fluidity, possibly leading to cell death. Rich in PUFA, lipoproteins are particularly sensitive to the action of ROS. Oxidized lipoproteins no longer correctly transport cholesterol. In addition, they are recognized by blood macrophages and accumulate inside them. The macrophages then take on the appearance of foam cells, which attach to artery walls. This is the mechanism by which oxidized lipoproteins contribute to increasing the risk of cardiovascular disease.

Recent studies have shown that ROS can also play a role at the molecular level. An example is their action on NF-κB, a B-lymphocyte-specific transcription factor. Maintained inactive in the cytoplasm, NF-κB can be induced in a wide variety of cell types by various factors, including cytokines, infectious agents, and also ROS acting as second messengers. Thioredoxin (TRX), a protein induced by oxidative stress, also increases the activity of NF-κB by modifying the redox regulation of glutathione (GSH). Once activated, NF-κB migrates to the nucleus of the cell, where it can transactivate target genes. It is thus involved in the synthesis of many mediators of the immune and inflammatory responses (cytokines, complement). Several viruses such as HIV also depend on NF-κB to replicate in the cell.

Xanthohumol (2′,4′,4-trihydroxy-6′-methoxy-3′-prenylchalcone [CAS #6754-58-1]) is a prenylated chalcone (and prenylated flavonoid) from hops (Humulus Iupulus L.). Only relatively minute quantities of xanthohumol are available in hops. Therefore, the amount of xanthohumol present in beer is not effective in eliciting biological effects.

Because of the destructive effects of oxidative stress, there is a need in the art for anti-oxidant compounds that effectively reduce or prevent oxidative stress. Unfortunately, compounds exhibiting potent anti-oxidative properties in vitro often fail to effectively reduce oxidative stress in vivo, including quercetin (O'Reilly et al., Am J Clin Nutr (2001) 73, 1040-4) and polyphenols (Cerda et al., European Journal of Clinical Nutrition (2006) 60, 245-253). The present invention solves these and other needs in the art

### BRIEF SUMMARY OF THE INVENTION

It has been discovered that xanthohumol is surprisingly effective in reducing levels 15-F₂ₜ-IsoP in mammals. Thus, the present invention provides a completely new modality in the reduction and prevention of oxidative stress in mammals as well as treatment and prevention of diseases caused by oxidative stress.

In one aspect, methods are provided for reducing levels of 15-F₂ₜ-IsoP in a mammalian subject. The methods include administering to the mammalian subject an amount of xanthohumol sufficient to reduce levels of 15-F₂ₜ-IsoP in the mammalian subject.

In another aspect, methods are provided for reducing and/or preventing oxidative stress in a mammalian subject in a mammalian subject. The methods include administering to the mammalian subject an amount of xanthohumol sufficient to reduce levels of 15-F₂ₜ-IsoP in the mammalian subject.

In another aspect, the present invention provides a method of treating and/or preventing, a disease caused by oxidative stress in a mammalian subject in need thereof. The method includes administering to the mammalian subject an effective amount of xanthohumol.

### DETAILED DESCRIPTION OF THE INVENTION

### I. Reducing 15-F₂ₜ-IsoP Levels in Mammals

It has been discovered that administration of xanthohumol results in an unexpected decrease in the levels of 15-F₂ₜ-IsoP in mammals. The decrease is relative to the 15-F₂ₜ-IsoP levels prior to the administration, or in the absence of administration, of xanthohumol to the mammalian subject. Therefore the methods provided herein are useful in reducing levels of 15-F₂ₜ-IsoP in mammalian subjects. Because 15-F₂ₜ-IsoP is a well-known clinical biomarker of oxidative stress, the methods provided herein are also useful in reducing oxidative stress in a mammalian subject and/or preventing oxidative stress in a mammalian subject. The methods include administering to the mammalian subject an amount of xanthohumol sufficient to reduce levels of 15-F₂ₜ-IsoP in the mammalian subject.

In order to be effective in reducing levels of 15-F₂ₜ-IsoP, one skilled in the art will understand that the xanthohumol must be provided in a formulation that is sufficiently bioavailable to the mammalian subject. In some embodiments, the mammal is a human or domesticated mammalian animal, such as a cat, dog or horse. Thus, the present invention may be used to reduce oxidative stress in humans.

Levels of 15-F₂ₜ-IsoP may be measured using any appropriate method. In some embodiments, the levels of 15-F₂ₜ-IsoP are measured in the serum or urine of the mammalian subject using methods well known in the art. Methods of measuring 15-F₂ₜ-IsoP in the urine and serum of mammalian subjects are described in detail, for example, in U.S. Pat. Nos. 5,858,696 and 5,700,654.

In some embodiments, the levels of 15-F₂ₜ-IsoP are reduced by at least about 5%, 10%, 15%, 20%, 25%, 30%, 35%. 40%, 45%, 50%. 55%, 60%, 65%, 70%, 75%, 80%, 85%, or 90%. In other embodiments, the levels of 15-F₂ₜ-IsoP are reduced from about 5% to about 90%, about 5% to about 80%, about 5% to about 75%, 5% to about 65%, about 5% to about 55%, about 5% to about 45%, or about 5% to about 40%. In other embodiments, the levels of 15-F₂ₜ-IsoP are reduced from about 10% to about 90%, about 10% to about 80%, about 10% to about 75%, about 10% to about 65%, about 10% to about 55%, about 10% to about 45%, or about 10% to about 40%. In some related embodiments, the above levels are measured in the urine of the mammalian subject.

Thus, in some embodiments, a method is provided for reducing or preventing oxidative stress in a mammalian subject. The method includes administering to the mammalian subject an amount of xanthohumol sufficient to reduce levels of 15-F₂ₜ-IsoP in the urine of the mammalian subject by at least 10%, thereby reducing oxidative stress. In related embodiments, the levels of 15-F₂ₜ-IsoP in the urine are reduced in an amount set forth in the embodiments described in the preceding paragraph.

The methods of the present invention may be administered over a course of days, weeks, months, or years. In some embodiments, the reduction in 15-F₂ₜ-IsoP levels is observed within a day of a single administration of xanthohumol. In other embodiments, the reduction is observed after one, two, three, or four weeks treatment of xanthohumol (e.g. a once per day treatment). In other embodiments, the reduction is observed after one, two, three, or four months treatment of xanthohumol (e.g. a once per day treatment).

The amount of xanthohumol sufficient to reduce levels of 15-F₂ₜ-IsoP may be from about 0.5 mg to about 1000 mg, from about 1 mg to about 50 mg, from about 1 mg to about 20 mg, or about 3 mg to about 10 mg. In some embodiments, the dose of xanthohumol is about 1 mg, 3 mg, 5 mg, 10 mg, 20 mg, 30 mg, 40 mg, 50 mg, 60 mg, 70 mg, 80 mg, 90 mg, 100 mg, 250 mg, 500 mg, 750 mg, or 1000 mg. In still other embodiments, the dose of xanthohumol is about 5 mg. The xanthohumol is typically administered as a twice per day formulation, or more preferably a once per day formulation.

### II. Xanthohumol Formulations

Xanthohumol may be administered in any appropriate formulation providing xanthohumol in a bioavailable form. In some embodiments, the xanthohumol is provided in a water-soluble formulation. The water-soluble formulation typically includes a non-ionic surfactant in order to provide xanthohumol in a water-soluble form, and of course xanthohumol.

A “non-ionic surfactant,” as used herein, is a surface active agent that tends to be non-ionized (i.e. uncharged) in neutral solutions (e.g. neutral aqueous solutions). Useful non-ionic surfactants include, for example, non-ionic water soluble mono-, di-, and tri-glycerides; non-ionic water soluble mono- and di-fatty acid esters of polyethylene glycol;

non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFIL M-2125CS, LABRASOL, SOFTIGEN, and GELUCIRE.

In some embodiments, the non-ionic surfactant is a polyoxyl castor oil, or derivative thereof. Effective polyoxyl castor oils may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

In some embodiments, the water soluble formulation is a non-alcoholic formulation. A “non-alcoholic” formulation, as used herein, is a formulation that does not include (or includes only in trace amounts) methanol, ethanol, propanol or butanol. In other embodiments, the formulation does not include (or includes only in trace amounts) ethanol.

In some embodiments, the formulation is a non-aprotic solvated formulation. The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

In some embodiments, the water soluble formulation does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water soluble formulation does not include (or includes only in trace amounts) dimethyl sulfoxide. Thus, in some embodiments, the water soluble formulation does not include DMSO or ethanol.

In still other embodiments, the water soluble formulation does not include (or includes only in trace amounts) a non-polar aprotic solvent. Non-polar aprotic solvents are aprotic solvents whose molecules exhibit a zero molecular dipole. Examples include hydrocarbons, such as alkanes, alkenes, and alkynes.

The water soluble formulation of the present invention includes formulations dissolved in water (i.e. aqueous formulations).

In some embodiments, the water soluble formulation consists essentially of a xanthohumol and a non-ionic surfactant. A “water soluble formulation consisting essentially of xanthohumol and a non-ionic surfactant” means that the formulation includes a xanthohumol, a non-ionic surfactant, and optionally additional components widely known in the art to be useful in neutraceutical formulations, such as preservatives, taste enhancers, buffers, water, etc. A “water soluble formulation consisting essentially of a xanthohumol and a non-ionic surfactant,” as used herein, does not include components that would destroy the novelty and inventiveness of the formulation.

### III. Diseases Caused by Oxidative Stress

In some embodiments, the present invention provides a method of treating, or preventing, a disease caused by oxidative stress in a mammalian subject in need thereof. The method includes administering to the mammalian subject an effective amount of xanthohumol. The effective amount of xanthohumol is an amount sufficient to reduce levels of 15-F₂ₜ-IsoP in the mammalian subject and result in treatment and/or prevention of the subject disease. Amounts sufficient to reduce levels of 15-F₂ₜ-IsoP in the mammalian subject are discussed in detail above. The amount administered to the subject will depend on the type and severity of the disease, the amenability of the disorder to respond to xanthohumol, and on the characteristics of the individual and their metabolic ability to respond to xanthohumol, such factors including general health, age, sex, body weight and tolerance to xanthohumol.

Diseases caused by oxidative stress include, for example, inflammation, infection, atherosclerosis, hypertension, cancer, radiation injury, neurological disease, neurodegenerative disease, ischemia/reperfusion injury, aging, wound healing, glutathione deficiency, acquired immunodeficiency syndrome, sickle cell anemia, and diabetes mellitus. In some embodiments, the disease caused by oxidative stress is a neurological disease, a neurodegenerative disease, or sickle cell anemia.

With regard to inflammation, oxidative stress results in increased immune system activity, which leads to inflammation, recruitment of more immune cells, and release of cytokines and acute phase proteins that further exacerbate the stress on the body. In conditions where there is excessive free radical production or infection (e.g. AIDS), there is a severe alteration of interleukin-2 (IL-2) production, which secondarily occurs due to glutathione (GSH) depletion. IL-2 is a glycoprotein, which is produced in response to mitogens and antigenic stimuli. Excessive oxidative stress results in amplified production of TNF-alpha and IL-6. IL-6 initiates and encourages the production of acute phase proteins such as c reactive protein, serum amyloid A protein, fibrinogen, and mannan-binding lectin. IL-1, IL-6, and TNF-alpha stimulate, for example, CRP synthesis by inducing hepatic gene expression, which triggers a variety of inflammatory responses and associated pathologies. CRP is also a mediator of the complement system, part of the innate immune response. The complement system provides further stimulus of TH1 and TH2 adaptive immune responses, which adds to the inflammatory response.

Neurological and neurodegenerative diseases include depression, obsessive-compulsive disorder, Alzheimer's, allergies, anorexia, schizophrenia, as well as other neurological conditions resulting from improper modulation of neurotransmitter levels or improper modulation of immune system functions, as well as behavioral disorders such as ADD (Attention Deficit Disorder) and ADHD (Attention Deficit Hyperactivity Disorder). Oxidative stress links diverse neuropathological conditions that include stroke, Parkinson's Disease, and Alzheimer's Disease and has been modeled in vitro with various paradigms that lead to neuronal cell death following the increased accumulation of reactive oxygen species. For example, immortalized neurons and immature primary cortical neurons undergo cell death in response to depletion of the anti-oxidant glutathione, which can be elicited by administration of glutamate at high concentrations.

A number of these diseases have ROS toxicity as a central component of their underlying mechanism of nerve cell destruction, including, but not limited to, amyotrophic lateral sclerosis (ALS, or Lou Gehrig's disease), Parkinson's disease, and Alzheimer's disease. For example, Alzheimer's disease is a neurodegenerative disorder associated with aging and cognitive decline. Amyloid beta peptide (1-42) is a primary constituent of senile plaques and has been implicated in the pathogenesis of the disease. Studies have shown that methionine residue 35 of beta (1-42) may plays a critical role in oxidative stress and neurotoxicity.

Additionally, oxidative stress is associated with the selective loss of dopaminergic neurons of the substantial nigra in Parkinson's disease (PD). The role of alpha synuclein as a potential target of intracellular oxidants has been demonstrated by identification of posttranslational modifications of synuclein within intracellular aggregates that accumulate in PD brains, as well as the ability of a number of oxidative insults to induce synuclein oligomerization.

There is considerable evidence which indicates that HIV infection and subsequently ARC/AIDS is by in large a free radically mediated disease. This analysis can be made indirectly as judged by the antioxidant levels in humans and their consequences on the immune system. One of those antioxidants, glutathione (GSH), is decreased as a result of HIV infecting the host. The GSH levels continue to decrease as the disease progresses through ARC and finally to AIDS. Micromolar changes in GSH levels have an untoward effect on the function of T lymphocytes. GSH shows a multiplicity of uses in the immune system. Thiol concentrations (e.g. GSH) regulate the replication of HIV genomic expression. Increasing the concentrations of thiols (GSH, NAC, GSE (glutathione ester)) in culture medium of U1 cell line (promonocytes) results in suppression of viral assembly, HIV reverse transcriptase production and viral replication.

Sickle cell anemia is a genetically determined disease. Analysis of sickle cell patients RBC (HbS) demonstrates a number of peculiarities of the membrane such as frozen spectrin shell of irreversibly sickled RBC, an abnormal orientation of the lipid bilayer phospholipids, deficient calcium-ATPase, a propensity for HbS RBCs to adhere to vascular endothelium, and oxidized thiol groups on the HbS molecule. Ischemic injury occurs to organs. Additional evidence of free radical damage to HbS is a deficiency of alpha-tocopherol, increased amounts of malondialdehyde, and abnormal group cross linking by malonadehyde. Superoxide anions can enter into erthrocytes via anion channels, resulting in the formation of methemoglobin and the ultimate lysis of erythrocytes. Sickle RBCs spontaneously generate sixty percent greater quantities of superoxide and approximately 75% more hydrogen peroxide when compared with controls. Superoxide dismutase is increased by about 50%, glutathione peroxidase and catalase were decreased by approximately 50% and 29% respectively. Glutathione and vitamin E levels were significantly reduced.

The present invention is not to be limited in scope by the exemplified embodiments, which are intended as illustrations of single aspects of the invention. Indeed, various modifications of the invention in addition to those described herein will become apparent to those having skill in the art from the foregoing description. Such modifications are intended to fall within the scope of the invention. Moreover, any one or more features of any embodiment of the invention may be combined with any one or more other features of any other embodiment of the invention without departing from the scope of the invention. References cited throughout this application are hereby incorporated by reference herein in their entirety for all purposes, whether previously specifically incorporated or not.

### IV. Examples

The examples below are meant to illustrate certain embodiments of the invention, and are intended to limit the scope of the invention.

Lucifer Yellow was purchased from Molecular Probes (Eugene, Oreg.). Hanks buffer and all other chemicals were obtained from Sigma-Aldrich (St. Louis, Mo.).

### Example 1

Water soluble compositions of xanthohumol were formulated containing the non-ionic surfactant macrogolglycerol hydroxystearate 40. By heating and stirring this polyoxyl castor oil with a powdered xanthohumol extract (containing in excess of 20% xanthohumol), a clear greenish viscous solution was formed containing dissolved xanthohumol (hereinafter referred to as “xanthohumol gel formulation”). The powdered xanthohumol extract consisted of 20% xanthohumol with no any alpha acids, beta acids, or 8-prenylnaringenin. The xanthohumol gel formulation consisted of macrogolglycerol hydroxystearate 40 (100 mL) and powdered xanthohumol extract (10 grams), representing a ratio of surfactant:prenylflavonoid of 10:1

Water was added to this viscous solution for dilution purposes with solubility being maintained.

An aqueous solution of solubilized xanthohumol was achieved by adding water to this viscous solution (hereinafter referred to as “aqueous xanthohumol formulation”). More specifically, the aqueous xanthohumol formulation was prepared by warming the xanthohumol gel formulation to warm water to form a clear aqueous solution of xanthohumol. This aqueous xanthohumol formulation did not have undesirable flavor. The aqueous xanthohumol formulation consisted of water (200 mL), macrogolglycerol hydroxystearate 40 (100 mL), and powdered xanthohumol extract (10 grams), representing a ratio of 20:10:1 for the water:surfactant:prenylflavonoid. The aqueous xanthohumol formulation was analyzed by HPLC and found to contain 0.6%, or 6 mg/mL xanthohumol.

### Example 2

The solubility of the powdered xanthohumol extract in pH 7.4 Hank's Balanced Salt Solution (10 mM HEPES and 15 mM glucose was compared to the xanthohumol gel formulation. At least 1 mg of powdered xanthohumol extract or 100 mg of xanthohumol gel formulation was combined with 1 mL of buffer to make a ≧1 mg/mL powdered xanthohumol extract mixture and a ≧1 mg/mL xanthohumol gel formulation mixture, respectively. The mixtures were shaken for 2 hours using a benchtop vortexer and left to stand overnight at room temperature. After vortexing and standing overnight, the powdered xanthohumol extract mixture was then filtered through a 0.45-μm nylon syringe filter (Whatman, Cat #6789-0404) that was first saturated with the sample.

After vortexing and standing overnight, the xanthohumol gel formulation mixture was centrifuged at 14,000 rpm for 10 minutes. The filtrate or supernatant was sampled twice, consecutively, and diluted 10, 100, and 10,000-fold in a mixture of 50:50 assay buffer:acetonitrile prior to analysis.

Both mixtures were assayed by LC/MS/MS using electrospray ionization against the standards prepared in a mixture of 50:50 assay buffer:acetonitrile. Standard concentrations ranged from 1.0 μM down to 3.0 nM. Results are presented in Table 1 below.

TABLE 1
Solubility of Xanthohumol in pH 7.4 Phosphate Buffer

| | Solubility (μM) | | |
|---|---|---|---|
| Test Article Identification | Rep 1 | Rep 2 | AVG |
| Powdered Xanthohumol | 0.40 | 0.81 | 0.61 |
| Extract | | | |
| Xanthohumol Gel | 1860 | 1700 | 1780 |
| Formulation | | | |

As shown in Table 1, the powdered xanthohumol extract and xanthohumol gel formulation gel showed average solubility values in pH 7.4 Hank's Balanced Salt Solution of 0.61 μM and 1780 μM, respectively.

### Example 3

The permeability of the xanthohumol gel through a cell-free (blank) filter that was 0.4 microns was studied in order to determine the non-specific binding and cell-free diffusion Pₐₚₚ of the xanthohumol gel formulation through the microporous 0.4 micron membrane. The xanthohumol gel formulation was assayed at the 2 μM xanthohumol concentration in Hanks buffer (Hanks Balanced Salt Solution (HBSSg) containing 10 mM HEPES and 15 mM glucose) at a pH of 7.4 in duplicate. Donor samples were collected at 120 minutes. Receiver samples were collected at 60 and 120 minutes. The apparent permeability coefficient, Pₐₚₚ, and percent recovery were calculated as follows:

Pₐₚₚ=(dCᵣ/dt)×Vᵣ/(A×C₀)

Percent Recovery=100×((Vᵣ×Cᵣ^{final})+(V_{d}×C_{d}^{final}))/(V_{d}×C₀)
Where:
dCᵣ/dt is the slope of the cumulative concentration in the receiver compartment versus time in μM s⁻¹.
Vᵣ is the volume of the receiver compartment in cm³.
V_{d} is the volume of the donor compartment in cm³.
A is the area of the cell-free insert (1.13 cm² for 12-well Transwell).
Cᵣ^{final} is the cumulative receiver concentration in μM at the end of the incubation period.
C_{d}^{final} is the concentration of the donor in μM at the end of the incubation period.
C₀ is the initial concentration of the dosing solution in μM.

Results of the non-specific binding assessment are presented in Table 2, which shows the permeability (10⁻⁶ cm/s) and recovery of Xanthohumol across the cell-free filter.

TABLE 2

| Xanthohumol Dosing | | |
|---|---|---|
| Solution Concentration (μM) | Pₐₚₚ (10⁻⁶ cm/s) | |
| (Average, N = 2) | A-to-B^{A} | Recovery (%)^{B} |
| Rep. 1: 2.31 | Rep. 1: 18.6 | Rep. 1: 95 |
| Rep. 2: 2.46 | Rep. 2: 17.1 | Rep. 2: 99 |
| AVERAGE: 2.39 | AVERAGE: 17.9 | AVERAGE: 97 |

^{A}A low rate of diffusion (<20 × 10⁻⁶ cm/s) through the cell-free membrane may indicate a lack of free diffusion, which may affect the measured permeability.
^{B}Low recoveries caused by non-specific binding, etc. would affect the measured permeability.

### Example 4

To test the permeability of xanthohumol across Caco-2 cell monolayers, Caco-2 cell monolayers were grown to confluence on collagen-coated, microporous, polycarbonate membranes in 12-well Costar Transwell® plates. Details of the plates and their certification are shown below in Table 3. The test article was also the aqueous xanthohumol formulation, and the dosing concentration was 2 μM in the assay buffer (HBSSg) as in the previous example. Cell monolayers were dosed on the apical side (A-to-B) or basolateral side (B-to-A) and incubated at 37° C. with 5% CO₂ in a humidified incubator. Samples were taken from the donor chamber at 120 minutes, and samples from the receiver chamber were collected at 60 and 120 minutes. Each determination was performed in duplicate. Lucifer yellow permeability was also measured for each monolayer after being subjected to the test article to ensure no damage was inflicted to the cell monolayers during the permeability experiment. All samples were assayed for Xanthohumol by LC/MS/MS using electrospray ionization. The apparent permeability (Pₐₚₚ), and percent recovery were calculated as described above. Xanthohumol permeability results are presented in Table 4, which shows the permeability (10⁻⁶ cm/s) and recovery of Xanthohumol across Caco-2 cell monolayers. All monolayers passed the post-experiment integrity control with Lucifer yellow Papp<0.8×10−6 cm/s.

TABLE 3

| Plates | TW12 |
|---|---|
| Seed Date | Jun. 6, 2006 |
| Passage Number | 63 |
| Age (Days) | 22 |

| Parameter | Value | Acceptance Criteria |
|---|---|---|
| TEER Value (Ω · cm²) | 468 | 450-650 |
| Lucifer Yellow Pₐₚₚ, ×10⁻⁶ cm/s | 0.13 | <0.4 |
| Atenolol Pₐₚₚ, ×10⁻⁶ cm/s | 0.30 | <0.5 |
| Propranolol Pₐₚₚ, ×10⁻⁶ cm/s | 20.65 | 15-25 |
| Digoxin (B-to-A)/(A-to-B) Pₐₚₚ Ratio | 16.57 | >3 |

TABLE 4

| | | Dosing Conc. | Percent | Pₐₚₚ | Efflux | Significant | Absorption |
|---|---|---|---|---|---|---|---|
| Test Article | Direction | (μM) | Recovery^{C} | (10⁻⁶ cm/s) | Ratio | Efflux^{B} | Potential^{A} |
| Xanthohumol | A-to-B | Rep. 1: 2.07 | Rep. 1: 30 | Rep. 1: 0.94 | 2.1 | No | Medium |
| | | Rep. 2: 2.03 | Rep. 2: 28 | Rep. 2: 0.74 | | | |
| | | Average 2.05 | Average: 29 | Average: 0.84 | | | |
| | B-to-A | Rep. 1: 2.25 | Rep. 1: 81 | Rep. 1: 1.36 | | | |
| | | Rep. 2: 2.21 | Rep. 2: 80 | Rep. 2: 2.18 | | | |
| | | Average: 2.23 | Average: 81 | Average: 1.77 | | | |

^{A}Absorption Potential Classification:
Pₐₚₚ(A-to-B) ≧ 1.0 × 10⁻⁶ cm/s High
1.0 × 10⁻⁶ cm/s > Pₐₚₚ(A-to-B) ≧ 0.5 × 10⁻⁶ cm/s Medium
Pₐₚₚ(A-to-B) < 0.5 × 10⁻⁶ cm/s Low
^{B}Efflux considered significant if:
Pₐₚₚ(B-to-A) ≧ 1.0 × 10⁻⁶ cm/s and Ratio Pₐₚₚ(B-to-A)/Pₐₚₚ(A-to-B) ≧ 3.0
^{C}Low recoveries caused by non-specific binding, etc. can affect the measured permeability.

### Example 5

The following formulation was prepared as described below: purified xanthohumol 98% (5% by weight), propylene glycol (15% by weight), flavor (q.s.), povidone (10% by weight), and water (70% by weight).

Propylene glycol was warmed to about 100° F., and the purified xanthohumol (98%) is mixed until a clear yellowish solution is obtained. The warm mixture was slowly added to the water while mixing. Finally, povidone and flavor were added.

### Example 6

An aqueous solution of xanthohumol and macrogolglycerol hydroxystearate as prepared using the method in Example 1 was administered to eight human subjects with mildly elevated isoprostane levels. The dose of xanthohumol in the aqueous solution was 6 mg once per day at night for three weeks.

The aqueous solution was analyzed by HPLC to verify the content of xanthohumol per dose. Bottles were weighed before and after the study to monitor compliance. After 3 weeks, the 15-F₂ₜ-IsoP levels were normalized to creatine and measured using LC/MS (liquid chromatography/mass spectroscopy).

After three weeks, there was a 35.1% average decrease from the beginning average level of 15-F₂ₜ-IsoP to the finishing average level of 15-F₂ₜ-IsoP for the 8 human subjects. The median percentage decrease of 15-F₂ₜ-IsoP per human subject was 31.0%. The largest individual decrease in 15-F₂ₜ-IsoP of the group was 75.0%.

What is claimed is:

1. A method of reducing levels of 15-F₂ₜ-IsoP in a mammalian subject, said method comprising administering to said mammalian subject an amount of xanthohumol sufficient to reduce levels of 15-F₂ₜ-IsoP in said mammalian subject.

2. The method of claim 1, wherein levels of 15-F₂ₜ-IsoP are reduced in the urine of said mammalian subject.

3. The method of claim 2, wherein said amount of xanthohumol is sufficient to reduce levels of 15-F₂ₜ-IsoP by at least 10%.

4. The method of claim 2, wherein said amount of xanthohumol is sufficient to reduce levels of 15-F₂ₜ-IsoP by at least 20%.

5. The method of claim 2, wherein said amount of xanthohumol is sufficient to reduce levels of 15-F₂ₜ-IsoP by at least 30%.

6. The method of claim 2, wherein said amount of xanthohumol is sufficient to reduce levels of 15-F₂ₜ-IsoP by at least 40%.

7. The method of claim 1, wherein said xanthohumol is administered as a water-soluble formulation.

8. The method of claim 7, wherein said xanthohumol water-soluble formulation comprises: a) xanthohumol; and b) a non-ionic surfactant.

9. The method of claim 8, wherein said non-ionic surfactant is a non-ionic water soluble mono-, di-, or tri-glyceride; non-ionic water soluble mono- or di-fatty acid ester of polyethyelene glycol; non-ionic water soluble sorbitan fatty acid ester; polyglycolyzed glyceride; non-ionic water soluble triblock copolymers; or derivative thereof.

10. The method of claim 8, wherein said non-ionic surfactant is macrogolglycerol hydroxystearate.

11. The method of claim 1, wherein said amount of xanthohumol is at least 1 mg.

12. The method of claim 1, wherein said amount of xanthohumol is at least 3 mg.

13. The method of claim 1, wherein said amount of xanthohumol is at least 5 mg.

14. The method of claim 1, wherein said amount of xanthohumol is from 1 mg to 20 mg.

15. The method of claim 1, wherein said amount of xanthohumol is from 3 mg to 10 mg.

16. The method of claim 1, wherein said amount of xanthohumol is about 5 mg.

17. The method of claim 1, wherein said amount of xanthohumol is administered once per day.

18. The method of claim 1, wherein said amount of xanthohumol is administered once per day over a period of at least one week.

19. The method of claim 1, wherein said amount of xanthohumol is administered once per day over a period of at least two weeks.

20. The method of claim 1, wherein said amount of xanthohumol is administered once per day over a period of at least three weeks.

21. The method of claim 1, wherein said amount of xanthohumol is administered once per day, after dinner and before bedtime.

22. The method of claim 1, wherein said mammalian subject is a human subject.

23. A method of reducing or preventing oxidative stress in a mammalian subject, said method comprising administering to said mammalian subject an amount of xanthohumol sufficient to reduce levels of 15-F₂ₜ-IsoP in the urine of said mammalian subject by at least 10%, thereby reducing or preventing oxidative stress.

24. A method of treating or preventing a disease caused by oxidative stress in a mammalian subject in need thereof comprising administering to the mammalian subject an effective amount of xanthohumol.
